SEQUENCE LISTING (1 sequence)

SEQ ID NO: 1 - DNA, 18 bp, synthetic construct
  source 1..18: mol_type = other DNA
catcatcatcatcatcaa

(19) United States
(12) Patent Application Publication
(10) Pub. No.: US 2026/0216077 A1
(43) Pub. Date: Jul. 30, 2026

(54) GUV PRODUCTION METHOD

(71) Applicant: Katholieke Universiteit Leuven K.U. Leuven Research & Development, Leuven (BE)

(72) Inventors: Xavier CASADEVALL I SOLVAS, Leuven (BE); Jorik WAETERSCHOOT, Leuven (BE)

(21) Appl. No.: 19/159,128

(22) PCT Filed: Feb. 23, 2024
(86) PCT No.: PCT/EP2024/054610

§ 371 Date: Aug. 22, 2025

(30) Foreign Application Priority Data
Feb. 24, 2023 (EP) 23158443.4
Aug. 22, 2023 (EP) 23192658.5

(51) Int. Cl. A61K 9/1277

(52) U.S. Cl. CPC A61K 9/1277

(57) ABSTRACT

The invention relates to a method for making unilamellar vesicles using Pickering emulsions of aqueous droplets comprising lipids in oil wherein the Pickering emulsions is stabilized by amphiphilic nanoparticles. The invention further relates to the use of the method of the invention to form synthetic cells.

### FIELD OF THE INVENTION

The present invention relates to a method to make (Giant) Unilamellar Vesicles. The present invention also relates to synthetic cells comprising (Giant) Unilamellar Vesicles obtained with the method of the invention.

### BACKGROUND OF THE INVENTION

The basic unit of terrestrial life is the cell. These units consist of the compartmentalization of a set of specialized molecules able to catalyze a wide set of reactions (from information processing and replication to energy conversions). In the last decades, much scientific research has focused in recreating these key building blocks of life. An essential feature for the replication of cellular life processes in an artificial counterpart is the recreation of the outer cell membrane, which typically consists of a bilayer of diverse lipids. This membrane provides both protection and an adequate environment wherein essential active processes, mainly mediated by transmembrane proteins, can take place. Such artificial lipid bilayers can be mimicked by many models, but the most relevant ones for recreating artificial cells are unilamellar lipid vesicles in the size range of an eukaryotic cell (called giant unilamellar vesicles, GUVs). Currently, there are a wide range of protocols available for the generation of GUVs. The original methods consisted in thin film hydration and electroformation, which suffer from low encapsulation efficiencies and do not enable to tune the lamellarity and size of the final vesicles. Additionally electroformation in physiological, salty conditions is difficult, with the electric fields used causing lipid oxidation and the feasibility to incorporate only a limited amount of negatively charged lipids (<10%). Techniques like emulsion transfer, microfluidic jetting and double emulsion droplets enable high encapsulation efficiencies. These, though, are not without disadvantages: residues of the oil in which the lipids are dissolved are often present in the final GUVs influencing their properties. Furthermore, the manufacturing and operation of such microfluidic chips is oftentimes difficult and not always reproducible, so highly dependent on experience.

One of the most effective GUV production methods was, first introduced by Weiss et al. (Nat. Mater. 2018, 17, 89-96). This is based on the production, within an oil phase, of aqueous droplets that act as mechanical stabilizers for the nascent GUV. In this method, an aqueous droplet containing Large Unilamellar Vesicles (LUVs) is stabilized within a fluorinated oil (e.g., HFE 7500 or FC 40) with the help of two different fluorosurfactants, a triblock copolymer consisting of two perfluoropolyether (PFPE) chains linked to a polyethylene glycol (PEG) and a PFPE-carboxylic acid surfactant (i.e. Krytox 157 FSH). In the presence of Mg²⁺ ions, the negative charges of the carboxylic group on the Krytox surfactant destabilizes the LUVs contained within the droplet, resulting into their fusion at the droplet interface into one GUV with the size of the stabilizing droplet. This method provides high yields and easy encapsulation of biomolecules, which has enabled different applications such as: the creation of synthetic organelles, the encapsulation of cortical elements (i.e. microtubules and actine) into GUVs, the production of cytotoxic synthetic T-cells and the encapsulation of lyotropic liquid crystals. A key limitation of the use of surfactants is that these amphiphilic molecules can strongly interact with the encapsulated molecules in the droplets and, consequently, mediate undesired transport processes (via micellar or surfactant-association processes) to the outer oil phase. As a result the encapsulation efficiency of important cellular biomolecules is lowered. Furthermore, some biological molecules will lose structure and function, hindering GUV formation. It has, for example, been shown that it is not possible to encapsulate actin molecules without a second picoinjection step or without the use of specific pH sensitive lipids. Finally, releasing the GUVs from the droplets in which they are generated, requires the application of demulsifying strategies, such as microfluidic systems (which is expensive, complex and low throughput) or via the addition of chemical demulsifiers such as Perfluoro-1-octanol (PFO), which can interact with hydrophobic residues of proteins and destabilize them. All these issues hinder the production of biologically-relevant GUVs that may effectively act as artificial counterparts of actual cells.

The method of the invention, effectively makes use of fluorinated nanoparticles, or FNPs, to produce Pickering emulsions that are capable of destabilizing LUVs at the droplet interface resulting in the formation of GUVs. Compared to surfactant-based strategies for templating GUV formation at a droplet interface, the method of the invention presents several advantages. First, GUVs could be produced across a broader range of lipids (from four different compositions with negatively-, neutrally- and positively-charged lipids) and buffer conditions. Second, no biomolecules used in the production of these GUVs (lipids, oligonucleotides) or small molecules (dyes) were observed to leak from the aqueous phase into the continuous oil phase, demonstrating a higher capacity to compartmentalize and effectively utilize such biomolecules. Finally, GUVs could be efficiently released into an aqueous buffer by simple centrifugation, which effectively removed FNPs and eliminated the need to use potentially detrimental chemicals such as PFO.

### SUMMARY

The present invention relates to a method for making Unilamellar Vesicles comprising the steps of:
a. forming a Pickering emulsion of aqueous droplets in oil wherein said oil comprises amphiphilic nanoparticles and wherein said aqueous droplets, (i) are stabilized by a layer of said amphiphilic nanoparticles at the droplet surface and (ii) comprise lipids within the droplets' inner space; and,
b. incubating said Pickering emulsion for said lipids to coalesce into a lipid bilayer at the interface between the aqueous phase and said layer of amphiphilic nanoparticles, thereby making Unilamellar Vesicles.

In one embodiment, the method of the invention is for making Giant Unilamellar Vesicles (GUVs) and said aqueous droplets have a size ranging from 1 μm to 500 μm.

In one embodiment, the method of the invention further comprises after step (b) an additional step of
c. Releasing the Unilamellar Vesicles formed at step (b) from the Pickering emulsion into an aqueous solution.

In one embodiment, said releasing step (c) is performed by centrifugation.

In one embodiment, said aqueous droplets further comprise cations, preferably comprise divalent cations, more preferably comprise magnesium ions.

In one embodiment, said amphiphilic nanoparticles are silica nanoparticles.

In one embodiment, said amphiphilic nanoparticles have a size inferior to 420 nm.

In one embodiment, said amphiphilic nanoparticles have a size inferior to 205 nm.

In one embodiment, said amphiphilic nanoparticles are amphiphilic fluorinated particles and said oil is fluorinated oil.

In one embodiment, said amphiphilic fluorinated particles are silica nanoparticle fluorinated with fluoroalkylsilane.

In one embodiment, said amphiphilic fluorinated particles are fluorinated by reacting silica nanoparticles with an amount of fluoroalkylsilane superior to 2.54×10⁻⁴ mol/g of nanoparticle wherein said fluoroalkylsilane is the limiting reactant.

In one embodiment, said lipids comprised within the droplets' inner space are in Small Unilamellar Vesicles (SUVs), Large Unilamellar Vesicles (LUVs) and/or lipid nanodiscs.

In one embodiment, said lipids comprised within the droplets' inner space are suitable to form lipid bilayers.

In one embodiment, said lipids comprised within the droplets' inner space are selected from the group consisting of phospholipids, glycolipids, DOTAP, DGS-NTA, sphingolipids, diacylglycerol, lipids extracts from biological samples and derivatives and/or any combination thereof.

In one embodiment, said aqueous droplets further comprises at least one additional biological molecule.

The present invention also relates to a synthetic cell comprising at least one Unilamellar Vesicle made with the method of the invention and at least one biological molecule.

### DETAILED DESCRIPTION

The invention relates to a method for making Unilamellar Vesicles. As used herein, the term “Unilamellar Vesicle” refers to a vesicle, typically but not necessarily spherical, bounded by a single bilayer of amphiphilic lipids or a mixture of such lipids, containing aqueous solution inside the vesicle.

In one embodiment, the method of the invention is for making Giant Unilamellar Vesicles. As used herein, the term “Giant Unilamellar Vesicle” refers to a Unilamellar Vesicle of large size, typically having a size at or above 1 μm.

The method of the invention comprise a step of forming a Pickering emulsion of aqueous droplets in oil wherein said oil comprises amphiphilic nanoparticles, and wherein said aqueous droplets, (i) are stabilized by a layer of said amphiphilic nanoparticles at the droplet surface and (ii) comprise lipids within the droplets' inner space.

In a preferred embodiment, the method of the invention comprise a step of forming a Pickering emulsion of aqueous droplets in fluorinated oil wherein said fluorinated oil comprises amphiphilic fluorinated nanoparticles, and wherein said aqueous droplets, (i) are stabilized by a layer of said amphiphilic fluorinated nanoparticles at the droplet surface and (ii) comprise lipids within the droplets' inner space.

In one embodiment, the Pickering emulsion is formed by mixing or by using a microfluidic device. In one embodiment, the Pickering emulsion is formed by mixing.

Examples of microfluidic devices suitable to form aqueous droplets include, without being limited to those described in (Pan et al., ACS Appl. Mater. Interfaces 2014, 6, 21446-21453 and Pan et al., Anal. Methods 2017, 9, 4622-4629).

Suitable mixing technique for forming the Pickering emulsion include, without being limited to, those described in the example section such as high speed mixing and vortexing.

The size of the aqueous droplets within the Pickering emulsion can be adjusted depending on the desired size of Unilamellar Vesicles to be produced; Droplet size may be adjusted for example by choosing droplet production techniques and adjusting parameters thereof.

In one embodiment, wherein the method of the invention is for making Giant Unilamellar Vesicles, the aqueous droplets have a size, or largest dimension, superior or equal to 1 μm preferably superior or equal to 2 μm, 3 μm, 4 μm or 5 μm. In one embodiment wherein the method of the invention is for making Giant Unilamellar Vesicles, the aqueous droplets have a size, or largest dimension, ranging from 1 μm to 500 μm, preferably ranging from 1 μm to 450 μm, 1 μm to 400 μm, 1 μm to 350 μm, 1 μm to 300 μm, 1 μm to 250 μm, 1 μm to 200 μm, 1 μm to 150 μm, 1 μm to 100 μm, 1 μm to 90 μm, 1 μm to 80 μm, 1 μm to 70 μm, 1 μm to 60 μm, 2 μm to 60 μm, 3 μm to 60 μm, 4 μm to 60 μm or 5 μm to 50 μm.

In the context of present invention, aqueous droplet refers to a small volume of water, or dispersion of any substance in water, irrespective of its form. In one embodiment, the aqueous droplet is substantially ellipsoidal or substantially spherical, preferably the aqueous droplet is substantially spherical.

In the context of the invention, the aqueous droplets comprise lipids within the droplets' inner space. It is to be understood that said lipids are suitable to form lipid bilayers and provided in an amount sufficient for said lipids to form Unilamellar Vesicle in the method of the invention. The concentration of lipids may be adjusted for instance, and without being limited to, by accounting for the desired size of the Unilamellar Vesicles to be produced with the method of the invention using the method described in Weiss et al. (Nat. Mater. 2018, 17, 89-96).

In one embodiment said lipids are selected form the group consisting of, phospholipids, such as phosphatidylcholines, phosphatidylserines, phosphatidylethanolamine, and phosphatidylglycerol, glycolipids, DOTAP, 1,2-dioleoyl-sn-glycero-3-[(N-(5-amino-1-carboxypentyl)iminodiacetic acid) succinyl] (DGS-NTA), sphingolipids, diacylglycerol, lipids extracts from biological samples and derivatives and/or any combination thereof.

In one embodiment said lipids are selected form the group consisting of, phospholipids, such as phosphatidylcholines, phosphatidylserines, phosphatidylethanolamine, and phosphatidylglycerol, glycolipids, DOTAP, 1,2-dioleoyl-sn-glycero-3-[(N-(5-amino-1-carboxypentyl)iminodiacetic acid) succinyl] (DGS-NTA), sphingolipids and derivatives and/or any combination thereof.

In one embodiment, said lipids within the droplets' inner space are in liposomes. In one embodiment, said lipids within the droplets' inner space are in Small Unilamellar Vesicles (SUV), Large Unilamellar Vesicles (LUVs) and/or lipid nanodiscs. In the context of the invention, the droplet's inner space does not contain intact or living fungal, bacterial, animal or plant cells as a lipids source. Similarly it is to be understood that the droplets's inner space does not contain lipidic structures that are larger than the Unilamellar Vesicle to be formed using the method of the invention.

As used herein, the term “Large Unilamellar Vesicle” refers to a Unilamellar Vesicle of size superior or equal to 100 nm and inferior to 1 μm. As used herein, the term “Small Unilamellar Vesicle” refers to a Unilamellar Vesicle of size inferior to 100 nm.

In one embodiment, said aqueous droplets comprises cations. In one embodiment, said cations are divalent cations. In one embodiment, said cations are calcium ions and/or magnesium ions. In one embodiment, said cations are Ca²⁺ and/or Mg²⁺. In one embodiment, said cations are magnesium ions. In one embodiment, said cations are Mg²⁺.

In one embodiment, the concentration of said cation within the aqueous droplet is superior or equal to 1 mM, preferably superior or equal to 1.5 mM, 2.0 mM, 2.5 mM, 3.0 mM, 3.5 mM, 4.0 mM, 4.5 mM or 5 mM. In one embodiment, the concentration of said cation within the aqueous droplet is ranging from 1 mM to 100 mM, preferably is ranging from 1 mM to 95 mM, from 1 mM to 90 mM, from 1 mM to 85 mM, from 1 mM to 80 mM, from 1 mM to 75 mM, from 1 mM to 70 mM, from 1 mM to 65 mM, from 1 mM to 60 mM, from 1 mM to 55 mM, from 1 mM to 50 mM, from 1 mM to 45 mM, from 1 mM to 40 mM, from 1 mM to 35 mM, from 1 mM to 30 mM, from 1 mM to 25 mM or from 1 mM to 20 mM, more preferably is ranging from 2 mM to 20 mM, from 3 mM to 20 mM, from 4 mM to 20 mM or from 5 mM to 20 mM.

In a preferred embodiment, the oil is fluorinated. This embodiment is particularly advantageous when fluorinated nanoparticles are used in the method of the invention.

Example of fluorinated oils that can be used in the context of the invention include, without being limited to Novec™ HFE-7500 (CAS #: 297730-93-9), Fluorinert™ FC-40 oil (CAS #: 86508-42-1), Perfluorohexane (CAS #355-42-0), Perfluoromethyldecalin (PFMD—CAS #51294-16-7) and the like.

In one embodiment, said fluorinated oil is selected from the group comprising Novec™ HFE-7500 (CAS #: 297730-93-9), Fluorinert™ FC-40 oil (CAS #: 86508-42-1) and mixture thereof.

In one embodiment, the amphiphilic nanoparticles are fluorinated. This embodiment is particularly advantageous when fluorinated oil is used in the method of the invention.

In the context of the invention, amphiphilic nanoparticles, preferably amphiphilic fluorinated nanoparticles, are present within the oil, preferably the fluorinated oil, so as to act as an emulsifier, thereby allowing the formation of the Pickering emulsion wherein the aqueous droplets are stabilized within the (fluorinated) oil, by said amphiphilic (fluorinated) nanoparticles, accumulating at the interface between the aqueous phase and the (fluorinated) oil phase.

In one embodiment, the nanoparticles are ceramic nanoparticles, metal nanoparticles, polymeric nanoparticles, semiconductor nanoparticles, or a combination thereof. In one embodiment, nanoparticles are silica nanoparticles.

As used herein The term “silica nanoparticle(s)” refers to both nanoparticles with a silica surface and particles essentially made of silica. In one embodiment, said silica particles consist of, or consist essentially of, silica.

In one embodiment, the amphiphilicity of said amphiphilic nanoparticles is adjusted to allow their accumulation at the interface between the aqueous droplets and the oil phase. In one embodiment, the amphiphilicity of said amphiphilic nanoparticles is adjusted for said amphiphilic nanoparticles to form a layer of at the interface between the aqueous droplet and the oil. In one embodiment, the amphiphilicity of said amphiphilic nanoparticles is adjusted for said amphiphilic nanoparticles to stabilize the Pickering emulsion.

In one embodiment, the amphiphilic fluorinated nanoparticles comprises fluorinated group covalently bound to the surface of the nanoparticles. In one embodiment said fluorinated group is a fluoroalkyl. The term “fluoroalkyl”, as a group or part of a group, refers to an alkyl group, wherein one or more hydrogen atoms are each replaced with a fluorine. The term “alkyl” as a group or part of a group, refers to a hydrocarbyl group of formula CₙH₂ₙ₊₁ wherein n is a number greater than or equal to 1, with no site of unsaturation. Alkyl groups may be linear or branched and may be substituted. Generally, alkyl groups of this invention comprise from 1 to 18 carbon atoms, preferably from 2 to 14 carbon atoms, more preferably from 4 to 12 carbon atoms, more preferably from 6 to 10 carbon atoms.

In one embodiment, the amphiphilicity of said amphiphilic fluorinated nanoparticles is adjusted to allow their accumulation at the interface between the aqueous droplets and the fluorinated oil phase. In one embodiment, the amphiphilicity of said amphiphilic fluorinated nanoparticles is adjusted for said amphiphilic fluorinated nanoparticles to form a layer of at the interface between the aqueous droplet and the fluorinated oil. In one embodiment, the amphiphilicity of said amphiphilic fluorinated nanoparticles is adjusted for said amphiphilic fluorinated nanoparticles to stabilize the Pickering emulsion.

The amphiphilicity of silica nanoparticles may, for example and without being limited to, be adjusted by adjusting the degree of fluorination.

In one embodiment, the amphiphilic fluorinated nanoparticles are silica nanoparticles fluorinated to allow their accumulation at the interface between the aqueous droplets and the fluorinated oil phase. In one embodiment, the amphiphilic fluorinated nanoparticles are silica nanoparticles fluorinated for said amphiphilic fluorinated silica nanoparticles to form a layer of said nanoparticles at the interface between the aqueous droplet and the fluorinated oil. In one embodiment, the amphiphilic fluorinated nanoparticles are silica nanoparticles fluorinated to stabilize the Pickering emulsion.

In one embodiment, said amphiphilic fluorinated nanoparticles are silica nanoparticles fluorinated with a fluoroalkylsilane.

In one embodiment, said amphiphilic fluorinated nanoparticles are silica nanoparticles fluorinated by reacting, preferably by reacting completely, said silica particles with an amount of fluoroalkylsilane superior to 2.54×10⁻⁴ mol/g of nanoparticle, preferably superior or equal to 1.27×10⁻³ mol/g of nanoparticle, 2.53×10⁻³ mol/g of nanoparticle, 5.07×10⁻³ mol/g of nanoparticle, 7.60×10⁻³ mol/g of nanoparticle and wherein said fluoroalkylsilane is the limiting reactant.

In one embodiment, said amphiphilic fluorinated nanoparticles are silica nanoparticles fluorinated by reacting, preferably by reacting completely, said silica particles with an amount of fluoroalkylsilane inferior or equal to 0.4 mol/g of nanoparticle, preferably inferior or equal to, 0.3 mol/g of nanoparticle, 0.2 mol/g of nanoparticle, 0.1 mol/g of nanoparticle, 9×10⁻² mol/g of nanoparticle, 8×10⁻² mol/g of nanoparticle, 7×10⁻² mol/g of nanoparticle, 6×10⁻² mol/g of nanoparticle, 5×10⁻² mol/g of nanoparticle, 4×10⁻² mol/g of nanoparticle or 3×10⁻² mol/g of nanoparticle, more preferably inferior or equal to 2.53×10⁻² mol/g of nanoparticle and wherein said fluoroalkylsilane is the limiting reactant.

In one embodiment, said fluoroalkylsilane is selected from the group consisting of 1H,1H,2H,2H-perfluorooctyltriethoxysilane (CAS #51851-37-7-FOTS), Triethoxyfluorosilane (CAS #358-60-1), Triethoxy-1H,1H,2H,2H-tridecafluoro-n-octylsilane (CAS #51851-37-7), Trimethoxy (3,3,3-trifluoropropyl)silane (CAS #429-60-7), Triethoxy(1H,1H,2H,2H-nonafluorohexyl)silane (CAS #102390-98-7), Trimethoxy(1H,1H,2H,2H-tridecafluoro-n-octyl)silane (CAS #85857-16-5) Tricholoro(1H, 1H,2H,2H-tridecafluoro-n-octyl)silane (CAS #78560-45-9), Cholorodimethyl(3,3,4,4,5,5,6,6,7,7,8,8,8-tridecafluoro-n-octyl)silane (CAS #102488-47-1), Trimethoxy(1H,1H,2H,2H-hepatdecafluorodecyl)silane (CAS #83048-65-1), Triethoxy (pentafluorophenyl)silane (CAS #20083-34-5), Triethoxy [5,5,6,6,7,7,7-heptafluoro-4,4-bis(trifluoromethyl) heptyl]silane (CAS #130676-81-2) and derivatives and/or any combination thereof.

In one embodiment, said fluoroalkylsilane is 1H,1H,2H,2H-perfluorooctyltriethoxysilane (CAS #51851-37-7-FOTS).

The term nanoparticle is used herein in reference to particles that have a size, or largest dimension, preferably an average size or average largest dimension, inferior to 1 μm, preferably superior or equal to 1 nm and inferior to 1 μm. In one embodiment, the nanoparticles are substantially spherical. In such embodiment the term size or largest dimension refers to the particle's diameter.

In one embodiment, said amphiphilic nanoparticles have a size, or largest dimension, preferably an average size or average largest dimension, inferior or equal to 430 nm, preferably inferior or equal to 420 nm, 410 nm, 400 nm, 390 nm, 380 nm, 370 nm, 360 nm, 350 nm, 340 nm, 330 nm, 320 nm, 310 nm, 300 nm, 290 nm, 280 nm, 270 nm, 260 nm, 250 nm, 240 nm, 230 nm, 220 nm or 210 nm, more preferably inferior or equal to 205 nm, 200 nm, 190 nm, 180 nm, 170 nm, 160 nm, 150 nm, 140 nm, 130 nm, 120 nm or 110 nm, even more preferably inferior or equal to 100 nm.

In one embodiment, said amphiphilic fluorinated nanoparticles have a size, or largest dimension, preferably an average size or average largest dimension, inferior or equal to 430 nm, preferably inferior or equal to 420 nm, 410 nm, 400 nm, 390 nm, 380 nm, 370 nm, 360 nm, 350 nm, 340 nm, 330 nm, 320 nm, 310 nm, 300 nm, 290 nm, 280 nm, 270 nm, 260 nm, 250 nm, 240 nm, 230 nm, 220 nm or 210 nm, more preferably inferior or equal to 205 nm, 200 nm, 190 nm, 180 nm, 170 nm, 160 nm, 150 nm, 140 nm, 130 nm, 120 nm or 110 nm, even more preferably inferior or equal to 100 nm. In embodiments wherein said amphiphilic nanoparticles are fluorinated, (average) size or (average) largest dimensions means herein (average) size or (average) largest dimensions after nanoparticles' fluorination.

In one embodiment, said amphiphilic nanoparticles have a size, or largest dimension, preferably an average size or average largest dimension, ranging from 20 nm to 430 nm, preferably ranging from 20 nm to 420 nm, from 20 nm to 410 nm, from 20 nm to 400 nm, from 20 nm to 390 nm, from 20 nm to 380 nm, from 20 nm to 370 nm, from 20 nm to 360 nm, from 20 nm to 350 nm, from 20 nm to 340 nm, from 20 nm to 330 nm, from 20 nm to 320 nm, from 20 nm to 310 nm, from 20 nm to 300 nm, from 20 nm to 290 nm, from 20 nm to 280 nm, from 20 nm to 270 nm, from 20 nm to 260 nm, from 20 nm to 250 nm, from 20 nm to 240 nm, from 20 nm to 230 nm, from 20 nm to 220 nm or from 20 nm to 210 nm, more preferably ranging from 20 nm to 205 nm, from 20 nm to 200 nm, from 20 nm to 190 nm, from 20 nm to 180 nm, from 20 nm to 170 nm, from 20 nm to 160 nm, from 20 nm to 160 nm, from 20 nm to 150 nm, from 60 nm to 140 nm, from 50 nm to 130 nm, from 40 nm to 120 nm, from 30 nm to 110 nm or from 20 nm to 100 nm.

In one embodiment, said amphiphilic fluorinated nanoparticles have a size, or largest dimension, preferably an average size or average largest dimension, ranging from 20 nm to 430 nm, preferably from 20 nm to 420 nm, from 20 nm to 410 nm, from 20 nm to 400 nm, from 20 nm to 390 nm, from 20 nm to 380 nm, from 20 nm to 370 nm, from 20 nm to 360 nm, from 20 nm to 350 nm, from 20 nm to 340 nm, from 20 nm to 330 nm, from 20 nm to 320 nm, from 20 nm to 310 nm, from 20 nm to 300 nm, from 20 nm to 290 nm, from 20 nm to 280 nm, from 20 nm to 270 nm, from 20 nm to 260 nm, from 20 nm to 250 nm, from 20 nm to 240 nm, from 20 nm to 230 nm, from 20 nm to 220 nm or from 20 nm to 210 nm, more preferably ranging from 20 nm to 205 nm, from 20 nm to 200 nm, from 20 nm to 190 nm, from 20 nm to 180 nm, from 20 nm to 170 nm, from 20 nm to 160 nm, from 20 nm to 160 nm, from 20 nm to 150 nm, from 60 nm to 140 nm, from 50 nm to 130 nm, from 40 nm to 120 nm, from 30 nm to 110 nm or from 20 nm to 100 nm. In embodiments wherein said amphiphilic nanoparticles are fluorinated, (average) size or (average) largest dimension means herein (average) size or (average) largest dimensions after nanoparticles' fluorination.

The method of the invention comprises, after the step of forming a Pickering emulsion, a step of incubating said Pickering emulsion for said lipids to coalesce into a lipid bilayer, preferably a single lipid bilayer, at the interface between the aqueous phase and the layer of amphiphilic (fluorinated) nanoparticles.

In one embodiment, the duration of the incubation is to allow said lipids to coalesce into a lipid bilayer, preferably a single lipid bilayer, at the interface between the aqueous phase and the layer of amphiphilic (fluorinated) nanoparticles. This duration may be determined by, for example and without being limited by, using labelled lipids and monitoring the formation of a lipid layer at the droplets' surface as described in the example section.

In one embodiment, the duration of the incubation step is of at least 1 hour, preferably at least 2 hours, at least 3 hours, at least 4 hours, at least 5 hours, at least 6 hours, at least 7 hours, at least 8 hours or more.

In one embodiment, the temperature during the incubation step is inferior or equal to 50° C., preferably inferior or equal to 45° C., 40° C., 35° C., 30° C., 25° C., 20° C., 15° C. or 10° C., more preferably inferior or equal to 5° C. In one embodiment, the temperature during the incubation step is superior 0° C.

It may be advantageous for further use of the (Giant) Unilamellar Vesicles produced by the method of the invention, to release, after the incubation step, the Unilamellar Vesicles formed during said incubation step from the Pickering emulsion, in an aqueous solution.

In one embodiment, the method of the invention comprises, after the incubation step, a step of releasing the (Giant) Unilamellar Vesicles formed during the incubation step from the Pickering emulsion into an aqueous solution.

In one embodiment said releasing step is performed by centrifugation. In one embodiment, said releasing step comprises centrifugating the Pickering emulsion containing (giant) Unilamellar Vesicles resulting from the incubation step.

In one embodiment, said releasing step comprises
(i) adding an aqueous solution to the Pickering emulsion containing Unilamellar Vesicles resulting from the incubation step; and,
(ii) centrifugating the solution obtained at step (i).

In one embodiment, the duration of the centrifugation is superior or equal to 15 minutes, preferably superior or equal to 20 minutes or 25 minutes, more preferably superior or equal to 30 minutes.

In one embodiment, the speed of the centrifugation is superior or equal to 17000 g, preferably superior or equal to 20000 g, 25000 g, 30000 g, 35000 g, 40000 g, 45000 g, 50000 g, 55000 g, 60000 g, 65000 g, 70000 g, 75000 g, 80000 g, 85000 g, 90000 g or 95000 g, more preferably superior or equal to 100000 g.

In one embodiment, the temperature during the centrifugation is inferior or equal to 50° C., preferably inferior or equal to 45° C., 40° C., 35° C., 30° C., 25° C., 20° C., 15° C. or 10° C., more preferably inferior or equal to 5° C. In one embodiment, the temperature during the centrifugation is superior 0° C.

The method of the invention may be used to form a synthetic cell, or artificial cell. In one embodiment, said a synthetic cell, or artificial cell, comprises at least one (Giant) Unilamellar Vesicles made, preferably directly obtained, with the method of the invention and at least one additional biological molecule.

The present invention also relates to a synthetic cell, or artificial cell, comprising at least one (Giant) Unilamellar Vesicles made, preferably directly obtained, with the method of the invention and at least one additional biological molecule.

When using the method of the invention, additional biological molecule(s) may be added at any step of the method of the invention (preferably in the aqueous solution used to form the aqueous droplet, within the aqueous phase and/or the dispersed lipids) and/or after the formation of the unilamellar vesicles.

In one embodiment, the aqueous droplet further comprises an additional biological molecule.

Biological molecule is used herein in reference to a molecule found in natural cells. Examples of biomolecules include, without being limited to, carbohydrates, lipids, proteins and nucleic acids. Example of Biological molecules that may be used in the context of the invention include, without being limited to,
whole or partial cell extract, including without being limited to, protein expression system (e.g. PURE system, New England Biolabs) and/or organelles;
nucleic acid chains, including, without being limited to, DNA, RNA, DNA origami structures, DNA logical networks and/or DNA nanopores;
transmembrane proteins, including without being limited to, GPCRs, transmembrane pores, cell adhesion molecules and/or cell receptors;
vesicle stabilising molecules, including without being limited to, hydrogels, self-assembling peptides, coacervates and/or cytoskeletal elements like actin, spectrin, microtubules and/or intermediate filaments; and/or
insulin, hemoglobin, growth factors, proteoglycans, perforins, hyaluronic acid and/or therapeutic biological product.

In one embodiment, the additional biological molecule is a transmembrane protein. In one embodiment the additional biological molecule is Band 3 anion transport protein (UniProt reference P02730).

### BRIEF DESCRIPTION OF THE DRAWINGS

FIG. 1 Panel A: Homogeneous LUV assembly, DOPC LUVs, 0 mM MgCl₂. Panel B: Patchy LUV assembly Egg LUVs, 2.5 mM MgCl₂. Scale bar 50 μm.

FIG. 2 Panel A: Overview image of droplets stabilized by rhodamine FNPs. Panel B Brightfield image of a droplet stabilized by rhodamine FNPs imaged in a cell counting chamber. Panel C: Rhodamine's Fluorescence image (confocal section) of the droplet in B. Panel D: Rhodamine's Fluorescence intensity profile along the white line drawn in Panel C. Scale bars 50 μm in A; 10 μm in panels B and C.

FIG. 3 Panel A: Homogeneous lipid distribution, Mix LUVs, 0 mM MgCl₂, 100 nm FNP (300 μl FOTS). Panel B: lipids assembled at droplet interface (“ring formation”), Plus LUVs, 0 mM MgCl₂·, 100 nm FNP (300 μl FOTS). Scale bars 50 μm.

FIG. 4 Panel A: Droplets stabilized with 3.6 wt % Krytox 1.4 wt % RAN with no leakage, DOPC LUVs 0 mM MgCl₂. B: Droplets stabilized with 3.6% Krytox 1.4% RAN with leakage, DOPC LUVs 7.5 mM MgCl₂. Scale bars 50 μm.

FIG. 5 GUVs released under different conditions. Panel A: DOPC 0 mM MgCl₂. Panel B: Mix 5 mM MgCl₂. Panel C: Egg 5 mM MgCl₂. Panel D: Plus 0 mM MgCl₂ Scale bars 50 μm.

FIG. 6 Panel A: Bright field (left) and fluorescence (right) images of DOPC (above—0 mM MgCl₂) and Mix (below—5 mM MgCl₂) droplet-stabilized GUVs with rhodamine labelled FNPs. Panel B: Brightfield (left) and fluorescence (right) images of DOPC (above—0 mM MgCl₂) and Mix (below—5 mM MgCl₂) GUVs released from Rhodamine FNPs-stabilized droplets. Panel C: Boxplot of Rhodamine fluorescence intensity values measured in droplets, released GUVs and background measured both for DOPC and Mix lipids. Panel D: Negatively stained TEM image of released DOPC (0 mM MgCl₂) GUVs. Panel E: Negatively stained TEM images of released Mix (5 mM MgCl₂) GUVs. White circles indicates few remaining FNPs. Scale Bars 10 μm in A and B; 500 nm in D and E.

FIG. 7 Graph showing the ratio of average fluorescein fluorescence intensity inside the GUVs vs outside the GUVs over time with α-hemolysin in the GUV membrane (dashed line) and without α-hemolysin in the GUV membrane (solid line). Grey area indicates SD.

FIG. 8 Encapsulation of Atto 488 dye (Panels A-D) and short cy5 labelled oligonucleotide (Panels E-H) in DOPC GUVs (0 mM MgCl₂-Panels A, B, E and F) and Mix GUVs (5 mM MgCl₂—Panels C, D, G and H). In both cases, 100 nm FNPs fluorinated with 300 μl FOTS are used. Panels B and D: Atto488 fluorescence. Panels F and H: cy5 labelled oligonucleotide fluorescence. Panels A, C, E and G Liss rhodamine PE fluorescence. Scale bars: 5 μm.

FIG. 9 Panels A-B: Micrographs of GUVs produced with proteoliposomes from erythrocytes with the nanoparticle assisted method stained with FITC-anti band 3 Ab (A) or FITC-isotype Ab (B). Panels C-D: Micrographs of GUVs produced with proteoliposomes from erythrocytes with the surfactant method stained with FITC-anti band 3 Ab (C) or FIC-isotype Ab (D). Scale bars: 25 μm.

### EXAMPLES

The present invention is further illustrated by the following examples.

### Example 1: Material and Methods

### Chemicals

1,2-dioleoyl-sn-glycero-3phosphocholine (DOPC), 1-palmitoyl-2-oleoyl-glycero-3-phosphocholine (POPC), 1,2-dioleoyl-3-trimethylammonium-propane (chloride salt) (DOTAP), 1,2-Dioleoyl-sn-glycero-3-phopsho-(1′-rac-glycerol (sodium salt)) (DOPG), Egg phosphatidylcholine (EggPC), Egg phosphatidylglycerol (EggPG), cholesterol, glucose, sucrose, tris(hydroxymethyl)aminomethane (TRIS), TEOS (Tetraethyl orthosilicate), 28 wt % ammonia, (3-Amino propyl)triethoxysilane (APTES), Rhodamine B isothiocyanate, Perfluoro-1-octanol (PFO), Bovine serum albumin (BSA), Fluorescein sodium salt, Atto 488 (catalog number 41051-1 MG-F) and chloroform were from Sigma-Aldrich (US). 1H,1H,2H,2H-perfluorooctyltriethoxysilane (FOTS) was from Fluorochem (UK). 4 μm silica particles (catalog number SiO2-R-4.0) were from microparticles GmbH (Germany). RAN 008 (catalog number 008-FluoroSurfactant-50G referred to as RAN herein) from RAN Biotechnologie (US), Krytox 157 FSH (referred to as Krytox herein) was from Costenoble (Germany), HFE 7500 (also referred to as HFE herein) from 3M (US) or Fluorochem (UK). Absolute Ethanol, Acetone and isopropanol (IPA) from acros organics (Belgium). Uranyl acetate from Electron Microscopy Sciences (US). Recombinant Staphylococcus alpha Hemolysin protein from Abcam (UK). 5′ Cy5™ modified DNA-Oligo (of sequence SEQ ID NO. 1) was from Integrated DNA Technologies (US) (5′-/5Cy5/CAT CAT CAT CAT CAT CAA A-3′). 1,2-dioleoyl-sn-glycero-3-phosphoethanolamine-N-(lissamine rhodamine B sulfonyl) (Liss rhod PE-cat 810150C-Avanti polar lipids, US). CD233/Band3 (ext) BRIC 6 FITC test vial was from NHS blood and transplants. FITC Rat IgG1 was from BioLegend Europe NV. Erythrocytes were obtained from Red Cross Flanders.

### Methods

### Silica Nanoparticle Production

100 nm, 200 nm and 400 nm silica nanoparticles were produced via the Stöber method (Mahalingam et al., Langmuir 2004, 20, 11756-11762). In short 3.8 ml of TEOS was dropwise added to 114 ml ethanol and 5.7 ml of 28 wt % ammonia hydroxide. The solution was left stirring overnight. Ammonia was evaporated and particles were stored at 0.01 g/ml. Alternatively for the 200 nm particles 216 ml ethanol, 9.52 ml milli-Q water, 15.51 ml ammonia hydroxide 28 wt % and 8.79 ml of TEOS were combined. For the 400 nm particles 215.7 ml ethanol, 11.68 ml milli-Q water, 12.18 ml 28 wt % ammonia and 11.06 ml of TEOS. Particles were further modified by reaction with FOTS. 200 μl of 28 wt % ammonia solution, 10 ml of 0.01 g/ml silica particles and 300 μl of FOTS (if not mentioned otherwise) was added and the solution was left shaking overnight. Afterwards the particles solutions was transferred to oakridge centrifuge tubes and spinned down for 30 min at 15 000 g. Particles were resuspended in ethanol by sonicating for 10 min. This cleaning process was repeated 3 times. Finally ethanol was removed and replaced with HFE 7500. To remove any leftover ethanol particle solutions were desiccated for at least an hour. To make rhodamine (rhod) labelled NP: First a rhodamine labeled APTES was produced by mixing 800 mg of APTES and 7.66 mg of rhod B isothiocyanate overnight. 100 μl of the solution was mixed with 300 μl FOTS together with the particles as described earlier to produces the rhodB labelled particles.

### LUV Production

In the presented work we chose to work with four different lipid compositions, named DOPC, Mix, Egg and Plus (mol %: mol percentage). DOPC (99.5 mol % DOPC, 0.5 mol % liss rhod PE), Plus (45 mol % DOPC, 45 mol % POPC, 9.5 mol % DOTAP, 0.5 mol % Liss rhod PE), Mix (34.75 mol % DOPC, 34.75 mol % POPC, 15 mol % DOPG, 15 mol % Cholesterol, 0.5 mol % Liss rhod PE), Egg (80 mol % EggPC, 19.5 mol % EggPG, 0.5 mol % Liss rhod PE) DOPC was chosen as a simple zwitterionic lipid system. Mix and Egg have been shown to have a relatively high release efficiency (Göpfrich et al., ACS Synth. Biol. 2019, 8, 937-947 and Haller et al., Lab Chip 2018, 18, 2665-2674). Plus charged lipids were used to show that vesicles can be released from positively charged lipid compositions. LUVs were produced by first transferring the different lipid components in a glass vial with the help of glass Hamilton syringes. Chloroform was evaporated by flushing with nitrogen and desiccation for at least two hours. The dried lipid film was hydrated with sucrose buffer solution vortexed for 30 s and sonicated for 30 min. Afterwards samples were extruded nine times through a 50 nm polycarbonate membrane with an Avanti Polar Lipid extruder set (catalog number. 610000, Avanti Polar Lipid, US).

LUV samples were stored in the fridge and used within three days of the extrusion process.

As an alternative to LUV, lipid nanodiscs can be used in the GUV production method. Such lipids nanodiscs can be obtained as follow: A 6% (wt/vol) Styrene Maleic Acid (SMA) stock solutions is prepared in 30 mM Tris 300 mM NaCl pH 7.5. SMA is slowly added to a solution containing LUVs or cells membrane fractions until a final concentration of 2% (wt/vol). Solution is then rocked for 2 h at 37° C. Insoluble fraction is removed by centrifuging at 100 000 g at 4° C. for 45 min.

Cell membrane fractions and be used to produce LUV, SUV or nanodiscs suitable for use in the methods of GUV production described herein.

### Dynamic Light Scattering (DLS) and Zetapotential Measurements

DLS and zetapotential measurements were performed with the zetasizer Nano ZSP (Malvern Panalytical, UK). For Zetapotential, samples were measured in a disposable capillary cell (DTS1070, Malvern Panalytical, UK) at 25° C. DLS samples were measured in a quartz cuvette (ZEN 2112, Malvern Panalytical, UK) at 173° angle (25° C.). Each sample was measured three times. Following material properties were used: EtOH (Refractive Index (RI): 1.287, Viscosity: 1.074 cP, Dielectric constant: 25.3), HFE 7500 (RI: 1.290, Viscosity: 1.240 cP, Dielectric constant: 5.8), buffer/PBS (properties of pure water at 25° C. were used, RI: 1.330 Viscosity: 0.887 cP), Lipids (RI: 1.450), Silica particles (RI: 1.540). Zetapotential of LUVs was measured in 1×DBPS.

### TEM

A 300 mesh cupper TEM grid was glow discharged for 15 s. 3.5 μl Sample was pipetted on and incubated for 5 min. For lipid containing samples: 30 μl of 1 vol % uranyl acetate was incubated on the grid for 1 min. Samples were blotted and imaged.

### Fluorinated Nanoparticles (FNP-Lipid Interaction)

4 μm (fluorinated) silica particles (microparticles GmbH, Germany) were incubated with the different LUV samples. To fluorinate the 4 μm silica particles 1 ml of commercial particle solutions was washed 3 times in ethanol by centrifuging at 1000 g for 5 min. Particles were redispersed with 5.37 ml ethanol and 153.6 μl 28 wt % ammonium hydroxide. 10 μl of FOTS was added and samples were left overnight shaking. Fluorinated particles were washed 3 times with ethanol. For the lipid incubation 25 μl of particles were washed 3 times with the respective buffer solutions by centrifuging at 1000 g for 5 min. These particles were transferred to 100 μl of 2 mM LUV solution in sucrose buffer and incubated for at least 3 h on a rotator.

### Droplet Production

Nanoparticle-stabilized droplets were produced by mixing 500 μl of the LUV-containing aqueous phase (2 mM lipids in sucrose buffer) and 250 μl of 0.1 g/ml fluorinated nanoparticle solution in 750 μl HFE 7500 with an ultra-turrax T-25 disperser (VWR international, US) at 24 000 rpm for 20 s. Droplets were stored overnight in the fridge at 4° C. RAN/krytox-stabilised droplets were produced by mixing 50 μl of the LUV-containing aqueous phase (sucrose buffer) with 100 μl of the surfactant mixture with a benchtop vortexer for 30 s. After vortexing a white droplet layer is formed on top of the oil phase. 2 different surfactant mixtures of RAN and krytox were prepared in HFE 7500 oil: 3.6 wt % krytox 1.4 wt % RAN and 5 wt % krytox 1.4 wt % RAN. These were optimized to give the best possible release.

### GUV Release

GUVs formed in nanoparticle stabilized droplets were released by taking 50 μl of droplet solution adding 50 μl of glucose release buffer and centrifugation for 30 min at 4° C. at 100 000 g. Surfactant stabilized droplets were released by addition of 50 μl of glucose release buffer solution and 50 μl of a 20 vol % PFO solution. These droplets were stored in the fridge for three hours before imaging.

### Rhodamine FNP on GUV Surface

Fluorescence intensity of GUVs, before and after release was analyzed in at least 20 droplets and 20 GUVs for both the DOPC and MIX lipid compositions, droplets were formed with rhodamine linked fluorescent nanoparticles. These values were compared to the background value. Imaging parameters were kept constant during both droplet and GUV imaging (for these experiments LUVs were not stained with rhodamine).

### Electroformation

10 μl of a 1.5 mg/ml lipid in chloroform was deposited on the conductive side of an ITO coated slide (vesicle prep pro-Nanion Technologies, DE) which was afterwards dessicated overnight. A rubber ring was attached to the slide with some wax around the lipid solution. Lipids were hydrated with 250 μl of a 300 mM sucrose in water solution and a second ITO coated slide was deposited on top. The slide sandwich was inserted into the vesicle prep pro device (Nanion technologies, DE). 3 V was applied at a frequency of 5 Hz for 160 min at 36° C. Temperature was ramped from room temperature to final temperature over 30 min. Amplitude was first slowly increased from 0.1 V to 0.5 V for 30 min and then to 3V in 15 min. At end of the electroformation, voltage was ramped down to 0 V in 5 min. GUVs were collected and stored in the fridge for maximum 1 day. 500 μl of 300 mM glucose in water solution was added before GUVs were transferred to the imaging chambers.

### Imaging

Confocal Imaging was performed with the Nikon TiE A1R (Nikon, Japan). Droplets were imaged by transferring 8 μl of droplet solution into a cell counting chamber (kisker biotech, Germany). Single GUV samples were imaged in 16 well uncoated polymeric Ibidi slides (catalog number 81816-Ibidi GmbH, Germany). Each well contained 100 μl of glucose release buffer and 50 μl of GUV solutions. For high throughput experiments a perkin elmer cell carrier ultra 96 well plate (PerkinElmer, US) was used. This plate was coated with BSA by incubating 10 mg/ml of BSA in dPBS solution overnight. Afterwards all wells were washed with dPBS and distilled water and filled with 100 μl of glucose release buffer before and images were collected with the Operetta CLS High Content Analysis System (PerkinElmer, US) 20× water immersion lens. Per well 69 images were taken in confocal mode effectively imaging the whole well. All GUV solution were handled with extra wide orifice pipette tips to reduce applied shear forces. 4 μm silica particles were imaged by transferring 10 μl of the particle solution in 100 μl of clean sucrose buffer in an ibidi slide.

### FRAP

GUVs were left for half an hour to sink to the bottom of the Ibidi slide. The center plane of the GUVs was brought in focus of the 60× lens of the Nikon TiE A1R. Next a 5 μm bleaching spot was defined at the edge of the GUV. At least 5 pre bleaching images were collected afterwards the spot was bleached for 1-2 cycles at full laser power and recovery images were collected for 30 s. Image sequences were normalized using the built in function of the nikon software with background and reference point correction. Recovery time calculation were performed by the Nikon software. Next diffusion constants were calculated with equation 1 (Kang et al., Traffic 2012, 13, 1589-1600)

$$D = \frac{{r}_{n}^{2}}{{𝒯}_{\frac{1}{2}}} \qquad ( 1 )$$

With rₙ² as radius of the bleachspot and τ_{1/2} as recovery time. Average and standard deviation of at least 9 samples were calculated and compared for both electroformed and nanoparticle formed GUVs.

### Unilamelarity Assay

10 μl of GUV solution was transferred to 100 μl of glucose release buffer solution in an Ibidi slide. Sample was left for half an hour to sediment GUVs. 50 μl of a 70 μM fluoresceine buffer solution with or without alpha hemolysine. When alpha hemolysin was added it was to a final concentration of 225 nM. Samples were imaged for at least 15 min. Fluorescence intensity of at least 10 GUVs was analyzed over time correcting for the change in background fluorescence.

### Encapsulation

1 μl of a Cy5-oligo stock solution was added to 100 μl of sucrose buffer solution with DOPC or Mix LUVs (containing 0 mM MgCl₂ and 5 mM MgCl₂ respectively) and GUV were generated as above. Atto 488 was similarly encapsulated at a concentration of 5 μM in DOPC and Mix GUVs. In both cases, the GUV membrane was labelled using Liss rhod PE.

### Release Efficiency

For the Mix and the DOPC lipids the release efficiency was determined for all the different nanoparticles sizes and fluorination degrees. This experiment was performed by using the Operetta CLS High Content Analysis System as described in the imaging section. Every conditions was repeated five times. Images were segmented and the number of GUVs was counted with the nikon analysis software.

### Buffer Preparation;

Dubelcos phosphate buffer saline (DPBS—catalog number 14190144) was from Fisher Scientific (US). Tris buffer is 30 mM Tris at pH 7.5. Glucose release buffer is tris buffer with 300 mM Glucose. Lipids/LUV solutions are in sucrose buffer: Tris buffer with 300 mM Sucrose complemented with MgCl₂ when and as indicated in each subsection. Sucrose is not necessary for droplet and/or GUV formation but is used to increase the weight of GUVs to facilitate imaging. Fluoresceine buffer is Tris buffer with 300 mM Glucose and with 70 μM of fluoresceine sodium salt.

### Transmembrane Protein Incorporation

Proteoliposomes (PLs)-enriched with Band 3 anion transport protein were isolated from human erythrocytes (according to Desrames, et al., I. Biochim. Biophys. Acta-Biomembr. 2020, 1862, 183126). Briefly human RBC extract was diluted with DPBS in molar ratios 2:1 (erythrocyte: DPBS). Samples were centrifuged for 5 min at 1000 g (4° C.). This process was repeated three times. The pellets were redispersed in low ionic strength solution (LIS, 5 mM sodium phosphate, 0.1 mM EDTA, pH 8) and incubated at RT for 15 min. Next, samples were centrifuged at 27 000 g for 15 min at 4° C., and were washed two more times. The pellets were then redispersed in very low ionic strength buffer (VLIS, 0.3 mM sodium phosphate, 0.1 mM EDTA, pH 8) and incubated at 43° C. for 30 min followed by centrifugation at 17 000 g for 10 min at 4° C. The resulting ghosts were washed with saline buffer (50 mM sodium phosphate, 300 mM NaCl, pH 8) and sonicated for 10 min at RT before being passed 11 times through a 100 nm polycarbonate membrane with a mini extruder (Avanti). As a result, monodisperse red blood cell proteoliposomes were obtained. These proteoliposomes were encapsulated in FNP (100 nm, 7.58×10⁻³ mol FAS/g NPs) and surfactant (3.6% krytox and 1.4% RAN) stabilized droplets as described before in a 10 mM MgCl2 solution. These droplets were then released and washed with Ab solution, by first centrifuging at 12 000 g for 10 min with release buffer and then incubating with either FITC-conjugated anti-Band 3 antibody or a FITC tagged isotype antibody (as a negative control) for 30 min on ice in the dark with interval gentle mixing each 10 min. Finally, GUVs were washed with the release buffer followed by imaging.

### Example 2: Nanoparticles (NPs) Characterization

We produced three different sets of silica NPs of around 100 nm, 200 nm and 400 nm via the Stöber method (Mahalingam et al., Langmuir 2004, 20, 11756-11762). The diameters and polydispersity indexes (PDI) of the 100 nm, 200 nm and 400 nm silica NPs and silica NPs fluorinated (forming Fluorinated NPs or FNPs) with different amounts of FOTS measured by DLS is indicated in Table 1.

TABLE 1
(F)NP diameter and PDI (Mean from three measurements).

| | | | | amount of |
|---|---|---|---|---|
| desired | amount | measured | | FOTS |
| diameter | of FOTS | diameter | PDI | (mol/g of |
| (nm) | (μl) | (nm ± SD) | (± SD) | particle) |
| 100 nm | 0 μl | 87 ± 2 | 0.1 ± 0.02 | 0 |
| 100 nm | 1 μl | 4521 ± 1424 | 0.5 ± 0.2 | 2.53 × 10⁻⁵ |
| 100 nm | 10 μl | 605 ± 16 | 0.3 ± 0.04 | 2.53 × 10⁻⁴ |
| 100 nm | 50 μl | 94.8 ± 0.6 | 0.04 ± 0.02 | 1.27 × 10⁻³ |
| 100 nm | 100 μl | 92.5 ± 0.6 | 0.02 ± 0.01 | 2.53 × 10⁻³ |
| 100 nm | 200 pl | 92.6 ± 0.3 | 0.02 ± 0.02 | 5.07 × 10⁻³ |
| 100 nm | 300 μl | 89.3 ± 0.4 | 0.16 ± 0.01 | 7.60 × 10⁻³ |
| 100 nm | 500 μl | 93.4 ± 0.5 | 0.02 ± 0.01 | 1.27 × 10⁻² |
| 100 nm | 1000 μl | 93 ± 0.6 | 0.02 ± 0.01 | 2.53 × 10⁻² |
| 200 nm | 0 μl | 211 ± 1 | 0.03 ± 0.02 | 0 |
| 200 nm | 100 μl | 219 ± 3 | 0.01 ± 0.01 | 2.53 × 10⁻³ |
| 200 nm | 200 μl | 207 ± 2 | 0.03 ± 0.01 | 5.07 × 10⁻³ |
| 200 nm | 300 μl | 224 ± 4 | 0.07 ± 0.03 | 7.60 × 10⁻³ |
| 400 nm | 0 μl | 393 ± 7 | 0.04 ± 0.02 | 0 |
| 400 nm | 50 μl | 430 ± 10 | 0.20 ± 0.01 | 1.27 × 10⁻³ |
| 400 nm | 100 μl | 430 ± 6 | 0.12 ± 0.06 | 2.53 × 10⁻³ |
| 400 nm | 200 μl | 431 ± 4 | 0.08 ± 0.04 | 5.07 × 10⁻³ |

For 100 nm NP, particle size remains similar after fluorination except when fluorinated with low amounts of FOTS (i.e. the cases of 1 μl and 10 μl). In such cases, the FNPs remain too hydrophilic, which leads to their aggregation in HFE oil. After fluorination of 200 nm and 400 nm NPs, sizes of the FNPs also remained similar to that of non-fluorinated NPs. The amount of FOTS (resulting in different fluorination degree) used to fluorinate the 200 and 400 nm NPs indicated in Table 1 were chosen since these resulted in the most stable droplets. Lower fluorination resulted in FNPs aggregation in HFE oil, while higher fluorination resulted in aggregation of the FNPs in the ethanol medium during their production. For the 100 nm NPs a more diverse set of fluorination degrees was investigated to assess their behavior at very low and very high fluorination degrees. The mean diameter of 100 nm non-fluorinated NPs functionalized with Rhodamine was 122.4±0.8 nm with a PDI of 0.065±0.002. These values remain similar to that of (F) NPs. The DLS size measurements of the (F) NPs were confirmed by TEM. A negative zetapotential was measured for the 100 nm NPs of −26±1 mV (mean±SD, 3 measurements) which increased after the fluorination reaction with 300 μl of FOTS to −19±1 mV (mean±SD, 3 measurements) likely due to a reduction in the number of silanol groups and consequently, of the surface charges.

### Example 3: LUV Characterization

Table 2 shows LUV size distributions in the different LUV solution, which all have a similar peak around 130 nm, confirming that the extrusion process provides homogeneous lipid samples.

TABLE 2
LUV diameter and PDI (mean from three measurements).

| lipid | MgCl₂ concentration | Diameter | |
|---|---|---|---|
| composition | (mM) | (nm ± SD) | PDI ( ± SD) |
| DOPC | 0 | 140 ± 1 | 0.11 ± 0.01 |
| DOPC | 2.5 | 139 ± 1 | 0.094 ± 0.004 |
| DOPC | 5 | 142 ± 1 | 0.109 ± 0.009 |
| DOPC | 7.5 | 145 ± 0.9 | 0.12 ± 0.02 |
| DOPC | 10 | 145 ± 0.9 | 0.12 ± 0.02 |
| Mix | 0 | 132.4 ± 0.8 | 0.12 ± 0.01 |
| Mix | 2.5 | 121.2 ± 0.6 | 0.09 ± 0.02 |
| Mix | 5 | 129.1 ± 0.5 | 0.1 ± 0.01 |
| Mix | 7.5 | 131.6 ± 0.3 | 0.1 ± 0.02 |
| Mix | 10 | 133.1 ± 0.9 | 0.097 ± 0.003 |
| Egg | 0 | 138.1 ± 0.4 | 0.09 ± 0.01 |
| Egg | 2.5 | 113.1 ± 0.1 | 0.09 ± 0.01 |
| Egg | 5 | 119.6 ± 0.8 | 0.079 ± 0.005 |
| Egg | 7.5 | 119.3 ± 0.5 | 0.092 ± 0.008 |
| Egg | 10 | 123.8 ± 0.3 | 0.08 ± 0.02 |
| Plus | 0 | 144 ± 2 | 0.13 ± 0.01 |
| Plus | 2.5 | 129.9 ± 0.6 | 0.12 ± 0.02 |
| Plus | 5 | 124.5 ± 0.4 | 0.1 ± 0.01 |
| Plus | 7.5 | 131.4 ± 0.4 | 0.127 ± 0.006 |
| Plus | 10 | 123.6 ± 0.5 | 0.106 ± 0.009 |

The zetapotential of the four different LUV was determined. As expected both the Egg and the Mix have a negative zetapotential (−21±1 mV and −16±1 mV respectively. mean±SD. 3 measurements) due to the presence of negative PG lipids. DOPC a neutral zetapotential (0.4±1 mV. mean±SD. 3 measurements) and the Plus lipids a positive zetapotential (18±2 mV. mean±SD. 3 measurements) as result of DOTAP (which is positively charged).

### Example 4: (F) NP-Lipid Interaction

Interactions between silica surfaces and lipid systems like LUVs are well characterized. These include: van der Waals. hydration. double-layer. hydrophobic. thermal undulation and protrusion forces. Additionally, the eventual fusion of the LUVs interacting with a silica surface is also mediated by the vesicle-vesicle interactions (Seantier and Kasemo, Langmuir 2009, 25, 5767-5772; Kettiger et al., Biochim. Biophys. Acta-Biomembr. 2016, 1858, 2163-2170; Anderson et al., Langmuir 2009, 25, 6997-7005; Cremer and Boxer, J. Phys. Chem. B 1999, 103, 2554-2559; and Michel and Gradzielski, Int. J. Mol. Sci. 2012, 13 (9), 11610-11642).

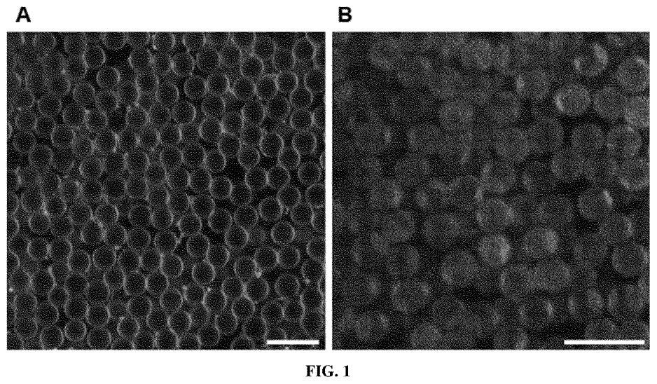

To investigate the interaction between vesicles and fluorinated silica surfaces. 4 μm fluorinated microparticles were incubated with the different LUV solutions used in this study. The fluorination of the microparticles was performed with 10 μl of FOTS. which is the maximum amount that still produced particles that could be incubated together with the lipid solutions without excessive aggregation. Lipid coverage results for bare silica microparticles and fluorinated microparticles. are summarized in Table 3 and Table 4 respectively. FIGS. 1A and B shows examples of both homogeneous and inhomogeneous lipid coverage.

TABLE 3
Lipid coverage of 4 μm non-fluorinated microparticles incubated
with different LUV solution.

| | | MgCl₂ concentration (mM) | | | | |
|---|---|---|---|---|---|---|
| | | 0 | 2.5 | 5 | 7.5 | 10 |
| lipid | DOPC | + | + | + | + | + |
| | Mix | + | + | + | + | + |
| | Egg | − | − | + | + | + |
| | Plus | + | + | + | + | + |

+indicates an homogeneous lipid coverage;
−indicates an inhomogeneous lipid coverage.

TABLE 4
Lipid coverage of 4 μm fluorinated microparticles incubated with
different LUV solution.

| | | MgCl₂ concentration (mM) | | | | |
|---|---|---|---|---|---|---|
| | | 0 | 2.5 | 5 | 7.5 | 10 |
| lipid | DOPC | + | + | + | + | + |
| composition | Mix | + | + | + | + | + |
| | Egg | + | + | + | + | + |
| | Plus | + | + | + | + | + |

+indicates an homogeneous lipid coverage;
−indicates an inhomogeneous lipid coverage.

For bare silica microparticles, LUVs assemble at the interface in all cases except for the 0 mM MgCl₂ and 2.5 mM MgCl2 Egg cases where a more inhomogeneous coating of the lipids is observed potentially due to a high negative zetapotential of Egg lipid LUVs and limited screening of charges as a results of low concentration of magnesium ions (Seantier and Kasemo, B. Langmuir 2009, 25, 5767-5772). Similar results were reported on glass surfaces as the result of the balance between attractive van der Waals forces and repulsive electrostatic forces under specific pH and buffer conditions, which led to the inhibition of the spreading of the lipids on such surfaces (Cremer and Boxer, Phys. Chem. B 1999, 103, 2554-2559). In the Mix case a higher zetapotential likely results in a lower electrostatic repulsive force which can be overcome by the zwitterionic attraction force, leading to a more homogeneous spreading of the lipids on the silica surface.

The interaction between DOPC LUVs and silica microparticles can be explained by a van der Waals attraction force. In DOPC the nitrogen of the headgroup is pointed more outwards than the phosphorus atom resulting in a dipole that interacts with the negative charge of the silica particle surface. This interaction is enlarged by the hydrogen bonding with the hydration layer (Seantier and Kasemo, B. Langmuir 2009, 25, 5767-5772; Kettiger et al., J. Biochim. Biophys. Acta-Biomembr. 2016, 1858, 2163-2170; Anderson et al., Langmuir 2009, 25, 6997-7005 and Helm et al., Science 1989, 246, 919-922). Similarly the Plus lipid mixture is likely attracted by the surface although here there will be an extra electrostatic attraction force as a result of the positive charge of the lipids. Given these more robust interactions, the coverage of these lipids on the silica microparticles was spread more evenly. With regards to fluorinated microparticles, the coverage with all lipid systems was observed to be homogeneous. This can be explained by a reduction of the number of hydroxyl groups due to the fluorination, also confirmed by an increase of the zetapotential, decreasing the electrostatic repulsion compared to the van der Waals attraction. These results indicate that the diverse lipids from our different LUVs can be effectively assembled on the surface of fluorinated silica particles.

### Example 5: Droplet Formation

### Droplet Formation

In all our experiments droplets were produced by mixing at high speed (23 000 rpm) an aqueous buffer with HFE 7500 oil containing FNPs, producing a polydisperse droplet population in the size range of interest (5-50 μm). Alternatively droplets can be produced by vortexing or on a microfluidic chip. Droplets produced with these alternative methods could also be efficiently stabilized by our different FNPs.

### Rhodamine FNPs

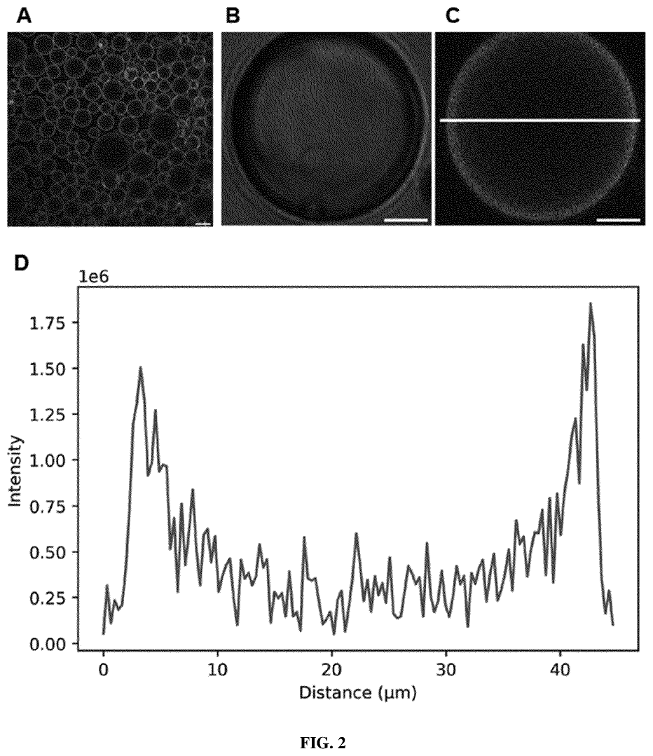

Rhodamine-linked FNPs were produced to confirm the distribution of the FNPs at a droplet interface. FIG. 2A illustrates a typical droplet population produced and imaged with these FNPs. FIG. 2B and FIG. 2C show magnified images of one of these droplets. The data show that rhodamine labelled FNPs can be used to produce and stabilize droplets. A characteristic fluorescence intensity profile across the equator of one of these droplets (FIG. 2D), clearly shows two intensity peaks at the droplet edges, where the droplet interface coincides with the focal plane of the confocal image. This indicates that these FNPs are self-assembling at the droplet interface, since the intensity gradually decays further from the droplet edges.

Droplet Formation with LUVs in FNP Stabilized Droplets

In the next steps LUVs from our different lipid mixtures were dispersed in several buffers. Table 5 shows droplets produced from these mixtures in HFE 7500 oil, stabilized by 100 nm FNPs fluorinated with 300 μl FOTS.

TABLE 5
Droplets with different LUV composition stabilized by 100 μm
microparticles fluorinated with 300 μl FOTS . . .

| | | MgCl₂ concentration (mM) | | | | |
|---|---|---|---|---|---|---|
| | | 0 | 2.5 | 5 | 7.5 | 10 |
| lipid | DOPC | + | + | + | + | + |
| composition | Mix | − | − | + | + | + |
| | Egg | − | − | + | + | + |
| | Plus | + | + | + | + | + |

+indicates lipids assembly at the droplet interface (ring formation);
−indicates condition in which lipids did not assemble at the droplet interface but remained homogeneously distributed.

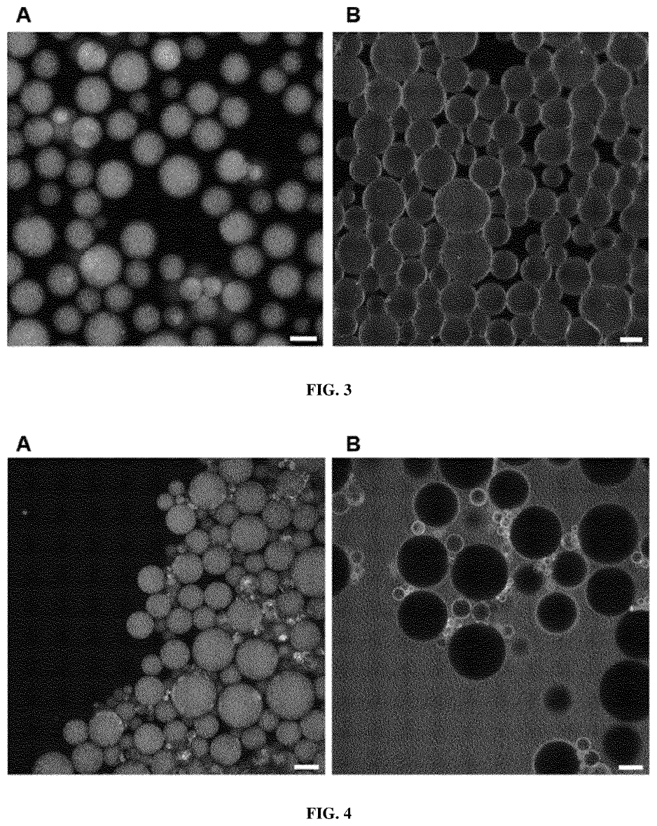

It can be seen that, for the two lowest MgCl₂ concentrations LUVs from Mix and Egg lipids did not assemble at the droplets interface (i.e. forming a fluorescent ring) but instead remained homogeneously distributed inside the droplets. FIG. 3A-B illustrates the difference between homogeneous distribution and ring formation. This was expected, since the repulsive forces between both negatively charged LUVs and FNPs could not be bridged at low magnesium concentrations. As a result, no GUVs could be produced under these conditions. This behavior is in contrast to the interaction of these lipids with the fluorinated 4 μm silica particles, indicating that the size of the fluorinated particles and/or their equilibration dynamics at the interface might be important factors affecting LUV destabilization. To evaluate the size effect of FNPs, we also tested FNPs of different sizes at diverse fluorination degrees.

Table 6 and Table 7 show that in all cases for 100 nm FNPs LUVs assembled at the droplet interface. This behavior is in line with the experiments in example 4 with the 4 μm microparticles in which in both fluorinated and non-fluorinated microparticles caused lipids from LUVs to assemble at the surface of the microparticles.

TABLE 6
Droplets formed with DOPC LUVs and 0 mM MgCl₂ with different
FNP sizes and fluorination degrees.

| | | amount of FOTS (ul) | | | | | | | |
|---|---|---|---|---|---|---|---|---|---|
| | | 1 | 10 | 50 | 100 | 200 | 300 | 500 | 1000 |
| particle | 100 nm | + | + | + | + | + | + | + | + |
| size | 200 nm | nd | nd | nd | − | − | − | nd | nd |
| | 400 nm | nd | nd | − | − | − | nd | nd | nd |

+indicates lipids assembly at the droplet interface (ring formation);
−indicates condition in which lipids did not assemble at the droplet interface but remained homogeneously distributed.
nd: not determined.

TABLE 7
Droplets formed with Mix LUVs and 5 mM MgCl₂ with different
FNP sizes and fluorination degrees . . .

| | | amount of FOTS (ul) | | | | | | | |
|---|---|---|---|---|---|---|---|---|---|
| | | 1 | 10 | 50 | 100 | 200 | 300 | 500 | 1000 |
| particle | 100 nm | + | + | + | + | + | + | + | + |
| size | 200 nm | nd | nd | nd | − | − | − | nd | nd |
| | 400 nm | nd | nd | − | − | − | nd | nd | nd |

+indicates lipids assembly at the droplet interface (ring formation);
−indicates condition in which lipids did not assemble at the droplet interface but remained homogeneously distributed.
nd: not determined.

In all cases of the 200 nm and 400 nm FNPs, most droplets did not present fluorescent rings (although some smaller droplets did display faint rings), indicating a general lack of capacity of these larger FNPs to attract and destabilize LUVs at the droplet interface. These results could potentially indicate that the relationship between the size of the LUVs and FNPs is of essence: since our 100 nm FNPs are smaller than the LUVs in these experiments, the latter might more easily bridge between FNPs, and the droplet interface in which the FNPs they are stabilized might act more like a homogeneous surface (i.e. like that of the 4 μm microparticles). In case of the 200 nm and 400 nm FNPs, the size of the LUV is much smaller compared to FNP size and, hence, the droplet surface might not act as a homogenous surface but more like a roughened silica surface, which has been was shown to inhibit the fusion of vesicles and spreading of their lipids into planar supported lipid bilayers (Cremer and Boxer, J. Phys. Chem. B 1999, 103, 2554-2559).

Droplet Formation with LUVs in RAN/Krytox Stabilised Droplets

To compare several aspects of our FNP-based method (i.e. leakage of biomolecules, droplet stability, release efficiency) with the standard droplet-stabilized strategy for GUV production (Weiss et al., Nat. Mater. 2018, 17, 89-96), droplets with the same lipid and buffer compositions were produced in HFE 7500 oil and stabilized by a combination of RAN and Krytox surfactants. In these cases (and also for the FNP cases) leakage of the LUVs into the oil phase was assessed by measuring the inner fluorescence of at least ten droplets and comparing it to the background fluorescence. For droplets stabilized by FNPs the outer fluorescence never became higher than the inner fluorescence. For surfactant-stabilized droplets, on the other hand, this happened in multiple conditions indicated in Table 8 and Table 9.

TABLE 8
Droplets with different LUV composition stabilized with 1.4% RAN and 5%
Krytox surfactant.

| | | MgCl₂ concentration (mM) | | | | |
|---|---|---|---|---|---|---|
| | | 0 | 2.5 | 5 | 7.5 | 10 |
| lipid | DOPC | − | +/leak | +/leak | +/leak | +/leak |
| composition | Mix | − | − | + | +/leak | +/leak |
| | Egg | − | − | − | +/leak | +/leak |
| | Plus | +/leak | +/leak | +/leak | +/leak | +/leak |

+indicates lipids assembly at the droplet interface (ring formation);
−indicates condition in which lipids did not assemble at the droplet interface but remained homogeneously distributed.
“+/leak” indicate condition were the fluorescence outside the droplet is higher than the fluorescence inside the droplet indicating serious leakiness.

TABLE 9
Droplets with different LUV composition stabilized with 1.4%
RAN and 3.6% Krytox surfactant.

| | | MgCl₂ concentration (mM) | | | | |
|---|---|---|---|---|---|---|
| | | 0 | 2.5 | 5 | 7.5 | 10 |
| lipid | DOPC | − | +/leak | +/leak | +/leak | +/leak |
| composition | Mix | − | − | − | +/leak | +/leak |
| | Egg | − | − | − | + | +/leak |
| | Plus | +/leak | +/leak | +/leak | +/leak | +/leak |

+indicates lipids assembly at the droplet interface (ring formation);
−indicates conditions in which lipids did not assemble at the droplet interface but remained homogeneously distributed.
“+/leak” indicate conditions where the fluorescence outside the droplet is higher than the fluorescence inside the droplet indicating serious leakiness.

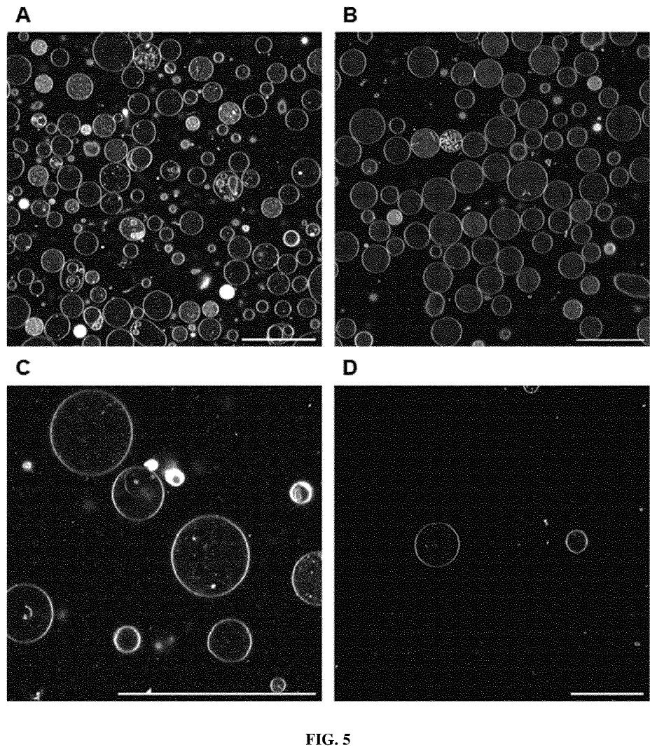

FIG. 4A-B illustrates the difference between non-leaking and leaking droplets. A high degree of leakage was observed in almost all cases were rings were formed, which are the essential conditions required for a sufficient interaction between Krytox and lipids in order to form GUVs. These results highlight an important disadvantage in the use of surfactants for droplet-stabilized GUV formation, as an important part of the biological elements contained in the droplets are lost into the oil phase, hindering the efficient encapsulation of biologically-relevant species in the GUVs. This is unsurprising, since it has repeatedly been shown that fluorinated surfactants (and specially Krytox) tend to establish strong interactions with a wide range of molecules that lead to their effective removal from aqueous droplets, when these are dispersed within a fluorinated oil phase (Gruner et al., Nat. Commun. 2016, 7, 10392; DeJournette et al., Anal. Chem. 2013, 85, 10556-10564 and Baret, Lab Chip 2012, 12, 422-433).

### GUV Release

After the formation of FNP stabilized droplets, confirmation of GUV formation (rather than just simple accumulation of lipids at the interface) was sought via attempting the release of these structures into an aqueous buffer. Different methods to merge NP-stabilized drops proposed by Pan et al. (Anal. Methods 2017, 9, 4622-4629) were explored to effectively release GUVs from our FNP-stabilized droplets. A first technique was via electrocoalescence in bulk. This was performed in an Eppendorf tube with ring shaped outer aluminum foil electrode on the outside and a copper electrode on the inside the Eppendorf tube (both coated with PDMS to prevent electrolysis) at DC voltages (up to 200 V) and frequencies (up to 1 kHz) as proposed by Pan et al (Anal. Methods 2017, 9, 4622-4629). None of these conditions achieved droplet coalescence. Next, chemical breakup with Perfluoro-1-octanol (PFO), typically also used for breakup of surfactant-stabilised droplets, was attempted but, again, coalescence was not achieved. Subsequently, thermal droplet coalescence by freeze-thawing was tested. This technique afforded droplet coalescence but unfortunately no GUVs could be retrieved. Finally mechanical break-up by centrifugation was applied. First with the parameters proposed by Pan et al (Anal. Methods 2017, 9, 4622-4629) (17 000 g for 15 min) were used, But also here our droplets did not coalesce. To observe coalescence, the centrifugation speed had to be increased to 100 000 g for 30 min. The droplets used by Pan et al (Anal. Methods 2017, 9, 4622-4629) are larger, around 75 μm since they are produced by manual shaking. In our case droplets are produced by high speed mixing resulting in a size around (5-50 μm). Since smaller droplets typically are more stable than larger droplets (Basheva et al., Langmuir 1999, 15, 6764-6769), it is likely that in our case larger mechanical forces were necessary to disrupt the droplets interface and release their contents. With this technique it was possible to observe released GUVs in the cases where ring formation was observed inside the original droplets. For all our four lipid compositions we were able to release GUVs by centrifugation (see FIG. 5A-D). Release from droplets with the Mix and DOPC LUVs yielded in general more putative GUVs when compared to Egg lipids and the Plus lipids.

### Example 6: GUV Characterization

### Rhodamine FNP on GUV Surface

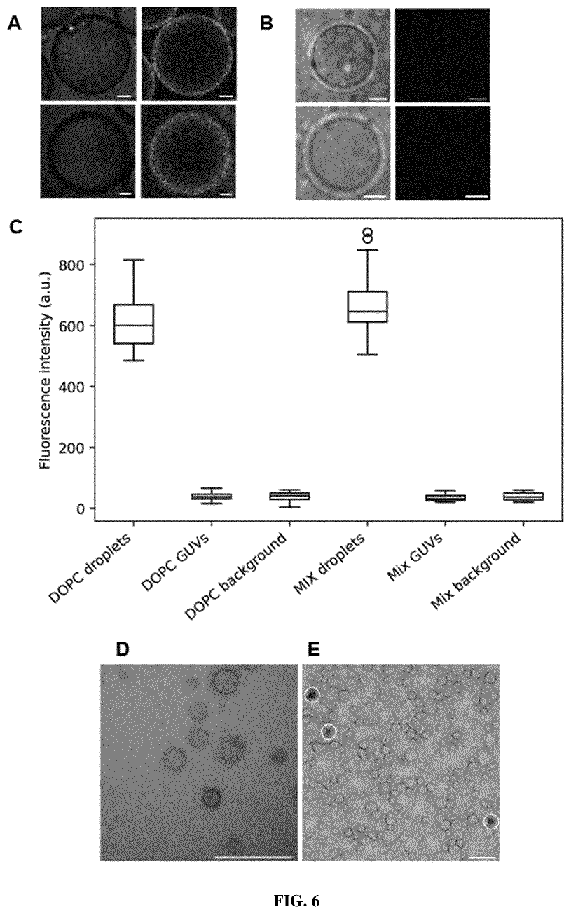

To elucidate whether the lipid assemblies released from our FNP-stabilized droplets were indeed GUVs, first we assessed if any FNPs still remained on these structures. We used rhodamine-labelled FNPs and assessed the fluorescence signal of rhodamine in both, FNP-stabilized droplets and released lipid assemblies, and this signal was compared to the background value (i.e. containing no rhodamine FNPs). As shown in FIG. 6A-C, the rhodamine fluorescence signal of released lipid assemblies is indistinguishable to that of the background values indicating that no FNPs are left on the putative GUV surface. To further confirm these results, TEM imaging (FIG. 6D-E) was performed on negatively stained samples in most samples no FNPs could be found, with only a few FNPs found in one of the samples. These experiments confirm both that the released lipid assemblies are not a complex structure consisting of FNPs and lipids and that centrifugation is an effective method for releasing these putative GUVs ensuring effective removal of FNPs at the same time.

### FRAP

FRAP experiments were performed on both electro- and FNP-formed GUVs of identical lipid composition. In Table 10 the data of the two different populations suggest that the formation method does not influence the diffusion characteristics of the lipid, providing evidence that, indeed, our FNP-based method produced GUVs.

TABLE 10
Diffusion constant (μm²/s) for the different GUV system produced either via
electroformation (elect.) or FNP-stabilized droplets (NP).

| DOPC | DOPC | Mix | Mix | Egg | Egg | Plus | Plus |
|---|---|---|---|---|---|---|---|
| elec. | NP | elec. | NP | elec. | NP | elec. | NP |
| 1.99 | 2.20 | 2.45 | 1.64 | 3.85 | 2.47 | 3.32 | 1.96 |
| 1.91 | 2.11 | 2.17 | 1.92 | 1.81 | 2.71 | 3.68 | 3.29 |
| 2.70 | 2.42 | 2.93 | 1.91 | 2.77 | 2.27 | 4.50 | 2.75 |
| 2.07 | 1.83 | 2.33 | 2.72 | 3.15 | 2.30 | 3.87 | 3.36 |
| 2.59 | 2.02 | 2.59 | 2.08 | 2.45 | 1.96 | 2.80 | 3.86 |
| 2.12 | 2.47 | 2.18 | 2.40 | 2.76 | 3.57 | 3.45 | 3.85 |
| 2.02 | 2.19 | 2.24 | 2.29 | 2.01 | 2.76 | 3.90 | 3.51 |
| 2.16 | 2.50 | 2.33 | 1.73 | 2.87 | 1.97 | 3.25 | 4.18 |
| 1.84 | 1.91 | 2.18 | 1.82 | 2.48 | 2.08 | 3.57 | 3.67 |
| 2.16 ± | 2.18 ± | 2.38 ± | 2.05 ± | 2.68 ± | 2.45 ± | 3.59 ± | 3.38 ± |
| 0.29 | 0.24 | 0.25 | 0.35 | 0.61 | 0.51 | 0.48 | 0.67 |

The last line indicates the mean ± SD for each condition.

Additionally these values are similar to what can be found in literature (Weiss et al., Nat. Mater. 2018, 17, 89-96; Schaich et al., Biochim. Biophys. Acta-Biomembr. 2020, 1862, 183359; Scherfeld et al., Biophys. J. 2003, 85, 3758-3768 and Kang et al., Traffic 2012, 13, 1589-1600).

### Unilamellarity Assay

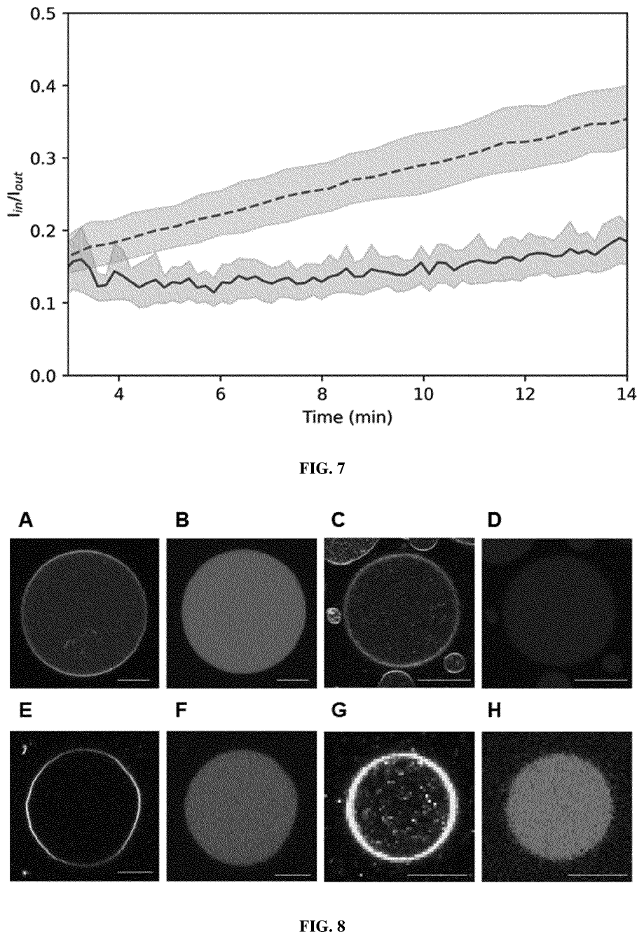

The unilamellarity of the GUVs was analysed with the alpha-hemolysin. This pore protein will assemble in the lipid bilayer enabling the leakage of a fluoresceine to the inside of the GUV upon addition to the outer phase. The pore protein can only incorporate in the outer bilayer as such no leakage can be seen in multilamellar vesicles would. In FIG. 7 it can be confirmed that in our case the fluorescence intensity increases upon addition fluoresceine to the outer buffer solution indicating that the formed GUVs have a unilamellar lipid bilayer.

### Encapsulation

Efficient encapsulation of Atto488 and a Cy5-labelled oligonucleotide was observed (FIG. 8A-H). This provides proof that encapsulation of both small molecules and biomolecules is possible within GUV formed in FNP-stabilized droplets.

### Release Efficiency

Finally, it is also important that GUVs formed via a droplet-stabilized method can be efficiently released from the droplet-oil system in which they were formed into an aqueous buffer in a simple manner. We, therefore, compared the Krytox surfactant-based method and our FNP-based method for the efficiency of releasing GUVs produced with both DOPC and Mix lipids, as illustrated in Table 11 and Table 12.

TABLE 11
number of DOPC GUVs released (mean of 5 repeats)
for different production methods and conditions.

| | MgCl₂ concentration (mM) | | | | |
|---|---|---|---|---|---|
| | 0 | 2.5 | 5 | 7.5 | 10 |
| 3.6% Krytox-1.4% RAN | 3 | 16414 | 1964 | 769 | 917 |
| 5% Krytox-1.4% RAN | 0 | 8 | 133 | 3 | 61 |
| 100 nm NP (300 μl FOTS) | 3515 | 911 | 4150 | 2141 | 4924 |

TABLE 12
number of MIX GUVs released (mean of 5 repeats)
for different production methods and conditions.

| | MgCl₂ concentration (mM) | | | | |
|---|---|---|---|---|---|
| | 0 | 2.5 | 5 | 7.5 | 10 |
| 3.6% Krytox-1.4% RAN | 143 | 237 | 918 | 1197 | 943 |
| 5% Krytox-1.4% RAN | 145 | 74 | 854 | 6792 | 7657 |
| 100 nm NP (300 μl FOTS) | 0 | 194 | 1528 | 2472 | 3451 |

For the DOPC lipids, the Krytox-based method was efficient only under one specific condition, whereas with FNP-based method was very efficient for almost all conditions. It is also important to highlight that GUVs can be produced with our method in the absence of MgCl₂, which might be essential for experiments where MgCl₂ must be avoided. In case of the Mix lipids release efficiencies were comparable between both methods (Gopfrich et al., ACS Synth. Biol. 2019, 8, 937-947 and Haller et al., Lab Chip 2018, 18, 2665-2674). Finally, as also shown in Table 13, we explored the effect of the degree of FNP fluorination on GUV formation.

TABLE 13
number of DOPC and MIX GUVs released (mean of 5 repeats)
for different production conditions using 100 nm FNPs.

| | | DOPC | MIX |
|---|---|---|---|
| Amount | 1 | 21 | 10 |
| of | 10 | 2063 | 75 |
| FOTS | 50 | 12721 | 5429 |
| (μl) | 100 | 9668 | 10600 |
| | 200 | 12642 | 21109 |
| | 500 | 14588 | 16497 |
| | 1000 | 5214 | 30506 |

Based on the DLS data we confirmed that when FNPs were fluorinated with less or equal than 10 μl of FOTS, FNPs aggregated in the fluorinated oil. This probably resulted in a inhomogeneous coverage of the droplets with FNPs, hindering effective GUV formation. A very high fluorination degree did not seem to have any influence on the GUV release. Finally, we also explored the effect of larger FNP sizes, as shown in Table 14 and Table 15.

TABLE 14
number of DOPC and MIX GUVs released (mean of 5 repeats)
for different production conditions using 200 nm FNPs.

| | | DOPC | MIX |
|---|---|---|---|
| Amount | 100 | 235 | 224 |
| of | 200 | 551 | 217 |
| FOTS | 300 | 2693 | 112 |
| (μl) | | | |

TABLE 15
number of DOPC and MIX GUVs released (mean of 5 repeats)
for different production conditions using 400 nm FNPs.

| | | DOPC | MIX |
|---|---|---|---|
| Amount | 50 | 336 | 3 |
| of | 100 | 182 | 135 |
| FOTS | 200 | 577 | 137 |
| (μl) | | | |

Except but for one case, no GUVs could be released efficiently. This is in line with the observation that for these larger FNPs, ring formation was typically not observed, except in droplets of smaller diameters.

### Example 7 Transmembrane Protein Incorporation

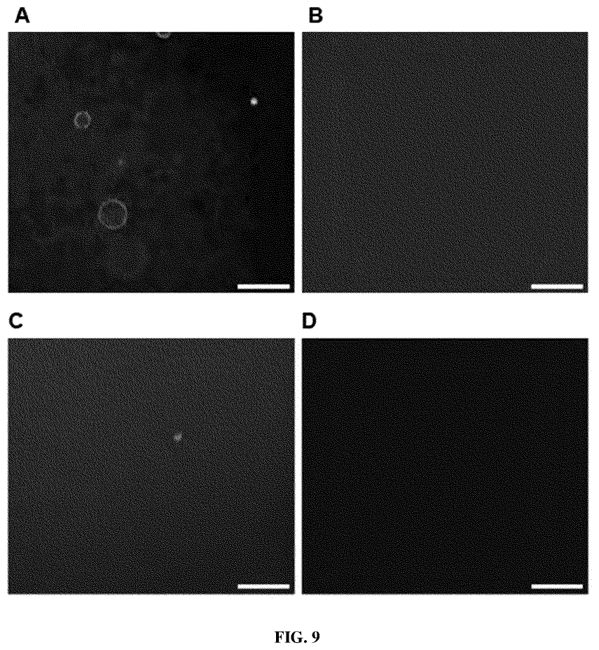

Besides encapsulation of biomolecules, to produce functional artificial cells it is of paramount importance to incorporate, in the lipid environment of the GUVs, functional transmembrane proteins. To assess the ability of our method to do so, band 3 anion transport protein, an erythrocyte transmembrane protein, was used as a model for complex membrane proteins. Small erythrocyte vesicles containing band 3 were encapsulated and subsequently destabilized inside a FNP-stabilized droplets, and the corresponding GUVs were released and stained with anti-band 3 conformational antibody (with a FITC-isotype antibody used as a control). In FIG. 9A-D it can be observed that the formed GUVs for erythrocyte fragments contained band 3 protein. Since the antibody used was a conformation-specific antibody for the ectodomain of band 3, at least a fraction of the incorporated band 3 was found both in its correct orientation and native folding configuration. GUVs were not visible after staining with FITC-isotype Ab indicating that the band 3 Ab specifically interacts with the protein and not with the lipid membrane. Band 3 incorporation with erythrocyte fragments using surfactants was not feasible since no GUVs were formed.

### Example 8 Conclusions

We present a novel method to more effectively harnesses the strategy of droplet-stabilized GUV formation. Our system, effectively makes use of fluorinated silica nanoparticles, or FNPs, to produce Pickering emulsions that are capable of destabilizing LUVs at the droplet interface resulting in the formation of GUVs. Compared to surfactant-based strategies for templating GUV formation at a droplet interface, our strategy presents several advantages. First, GUVs could be produced across a broader range of lipids (from four different compositions with negatively-, neutrally- and positively-charged lipids) and buffer conditions. Second, no biomolecules used in the production of these GUVs (lipids, oligos, dyes) were observed to leak from the aqueous phase into the continuous oil phase, demonstrating a higher capacity to compartmentalize and effectively utilize such biomolecules. Finally, GUVs could be efficiently released into an aqueous buffer by simple centrifugation, which effectively removed FNPs and eliminated the need to use potentially detrimental chemicals such as PFO. Release efficiencies between the two methods were comparable, if not slightly better in our case. Furthermore, we found that GUV formation with 100 nm FNPs was most effective with only a limited effect of the fluorination degree. The evidence provided, highlights that our method is a more robust alternative for the production of more complex, biologically relevant GUVs, with potential applications as artificial cells.

1. A method for making Unilamellar Vesicles comprising the steps of:
a. forming a Pickering emulsion of aqueous droplets in oil wherein said oil comprises amphiphilic nanoparticles and wherein said aqueous droplets, (i) are stabilized by a layer of said amphiphilic nanoparticles at the droplet surface and (ii) comprise lipids within the droplets' inner space; and,
b. incubating said Pickering emulsion for said lipids to coalesce into a lipid bilayer at the interface between the aqueous phase and said layer of amphiphilic nanoparticles, thereby making Unilamellar Vesicles.

2. The method according to claim 1, wherein said method is for making Giant Unilamellar Vesicles (GUVs) and wherein said aqueous droplets have a size ranging from 1 μm to 500 μm.

3. The method according to claim 1 or 2 further comprising after step (b) an additional step of
c. Releasing the Unilamellar Vesicles formed at step (b) from the Pickering emulsion into an aqueous solution.

4. The method according to claim 3, wherein said releasing step (c) is performed by centrifugation.

5. The method according to any one of claims 1 to 4, wherein said aqueous droplets further comprise cations, preferably comprise divalent cations, more preferably comprise magnesium ions.

6. The method according to any one of claims 1 to 5, wherein said amphiphilic nanoparticles are silica nanoparticles.

7. The method according to any one of claims 1 to 6, wherein said amphiphilic nanoparticles have a size inferior to 420 nm.

8. The method according to any one of claims 1 to 7, wherein said amphiphilic nanoparticles have a size inferior to 205 nm.

9. The method according to any one of claims 1 to 8, wherein said amphiphilic nanoparticles are amphiphilic fluorinated particles and wherein said oil is fluorinated oil.

10. The method according to claim 9, wherein said amphiphilic fluorinated particles are silica nanoparticle fluorinated with fluoroalkylsilane.

11. The method according to claim 9 or 10, wherein said amphiphilic fluorinated particles are fluorinated by reacting silica nanoparticles with an amount of fluoroalkylsilane superior to 2.54×10⁻⁴ mol/g of nanoparticle and wherein said fluoroalkylsilane is the limiting reactant.

12. The method according to any one of claims 1 to 11, wherein said lipids comprised within the droplets' inner space are in Small Unilamellar Vesicles (SUVs), Large Unilamellar Vesicles (LUVs) and/or lipid nanodiscs.

13. The method according to any one of claims 1 to 12, wherein said lipids comprised within the droplets' inner space are suitable to form lipid bilayers.

14. The method according to any one of claims 1 to 13, wherein said lipids comprised within the droplets' inner space are selected from the group consisting of phospholipids, glycolipids, DOTAP, DGS-NTA, sphingolipids, diacylglycerol, lipids extracts from biological samples and derivatives and/or any combination thereof.

15. The method according to any one of claims 1 to 14, wherein said aqueous droplets further comprises at least one additional biological molecule.
